(19) United States
(12) Patent Application Publication
(10) Pub. No.: US 2024/0241360 A1
(43) Pub. Date: Jul. 18, 2024

(54) IMAGING METHOD, FOCAL POSITION ADJUSTING METHOD, AND MICROSCOPE SYSTEM

(71) Applicant: SYSMEX CORPORATION, Kobe-shi (JP)

(72) Inventor: Kohei MURAKAMI, Kobe-shi (JP)

(73) Assignee: SYSMEX CORPORATION, Kobe-shi (JP)

(21) Appl. No.: 18/621,162

(22) Filed: Mar. 29, 2024

Related U.S. Application Data
(63) Continuation of application No. PCT/JP2022/015946, filed on Mar. 30, 2022.

(30) Foreign Application Priority Data
Sep. 30, 2021 (JP) 2021-162241

(51) Int. Cl. G02B 21/36

(52) U.S. Cl. CPC G02B 21/368; G02B 21/361; G02B 21/362

(57) ABSTRACT

An imaging method is disclosed that images specimen using a microscope system. An imaging method according to one or more embodiments may include determining candidate relative positions based on captured images of the specimen, the images being obtained by capturing images while changing a relative position between the specimen and a focal point of a light receiving optical system of the microscope system, determining a relative position for capturing among the candidate relative positions, and capturing an image of the specimen at the determined relative position.

### CROSS REFERENCE TO RELATED APPLICATIONS

This application is a continuation application of International Application No. PCT/JP2022/015946, filed on Mar. 30, 2022, which claims priority based on the Article 8 of Patent Cooperation Treaty from prior Japanese Patent Application No. 2021-162241, filed on Sep. 30, 2021, the entire contents of which are incorporated herein by reference.

### BACKGROUND

The disclosure relates to an imaging method of a specimen of a microscope system, a focal position adjusting method for adjusting a focal position of a microscope system, and a microscope system.

In the microscope system, the focal position with respect to specimen is adjusted prior to observation. Patent Literature 1 (International Publication No. 2018/158810) discloses an example of a focal position adjustment method. In the cell observation device using the holographic microscope disclosed in Patent Literature 1, a large number of phase images having different focal positions in a plurality of stages are created in advance. When the observer moves the knob of the slider arranged on the display image, the phase image of the focal position corresponding to the position of the knob is displayed in the image display frame on the display image. The observer confirms if the phase image of the image display frame is in the imaging state at each position of the knob while moving the knob of the slider. When the observer confirms that the phase image of the image display frame is in the imaging state by this operation, the observer operates the determination button on the display image to determine the focal position.

### SUMMARY

One or more embodiments relate to an imaging method of imaging a specimen using a microscope system. An imaging method according to one or more embodiments may include determining candidate relative positions based on captured images of the specimen, the image being obtained by capturing images while changing a relative position between the specimen and a focal point of a light receiving optical system of the microscope system, determining a relative position for capturing among the candidate relative positions; and capturing an image of the specimen at the determined relative position.

According to an imaging method according to one or more embodiments, candidate relative positions may be automatically determined based on captured images obtained while changing the relative position between specimen and the focal point of the light receiving optical system. A user may adjust the focal position only by selecting a relative position that a user considers appropriate from among the plurality of automatically determined candidate relative positions. Since it may not be necessary to find an appropriate relative position from captured images, it may be possible to adjust the focal position easily compared with technique in a related art.

One or more embodiments relates to a method of focal position adjusting that adjusts a relative position between a specimen and a focal point of a light receiving optical system. A method according to one or more embodiments may include determining candidate relative positions based on captured images of the specimen, the image being based on captured images while changing the relative position between specimen and the focal point of the light receiving optical system of the microscope system, and determining a relative position for capturing among the candidate relative positions.

According to the focal position adjusting method according to one or more embodiments, similarly to the imaging method described above, a user may adjust the focal position only by selecting a relative position that a user considers to be appropriate among a plurality of automatically determined candidate relative positions. Since it may not be necessary to find an appropriate relative position from captured images, it may be possible to adjust the focal position more easily than in the related art. Accordingly, the subsequent imaging of specimen may be performed in a state of being set to a desired relative position.

One or more embodiments relates to a microscope system that captures a specimen image. A microscope system according to one or more embodiments may include a specimen setting part that is placed specimen, an image sensor that captures an image of the specimen through a light receiving optical system, a driving unit that changes a relative position of a focal point of the light receiving optical system with respect to the specimen setting part, and a controller. In one or more embodiments, the controller may perform operations that include determining candidate relative positions based on specimen images captured by the image sensor while changing the relative position, determining a relative position for capturing from the relative positions, and capturing a specimen image at the relative by the image sensor.

According to the microscope system according to one or more embodiments, similarly to the imaging method described above, a user may adjust the focal position only by selecting a relative position that a user considers to be appropriate among a plurality of automatically determined candidate relative positions. Since it may not be necessary to find an appropriate relative position from captured images, it may be possible to adjust the focal position more easily than in the related art.

### BRIEF DESCRIPTION OF DRAWINGS

FIG. 1A is a diagram illustrating a perspective view of a configuration of a microscope system according to one or more embodiments.

FIG. 1B is a diagram illustrating a perspective view of a configuration of a microscope device according to one or more embodiments.

FIG. 2 is a schematic diagram illustrating an internal configuration of a microscope device according to one or more embodiments.

FIG. 3 is a block diagram illustrating a configuration of a microscope system according to one or more embodiments.

FIG. 4 is a flowchart illustrating processing performed by a controller of control device in the microscope system according to one or more embodiments.

FIG. 5 is a diagram illustrating a configuration of a screen displayed in a display according to one or more embodiments.

FIG. 6 is a diagram illustrating a configuration of a screen displayed in a display according to one or more embodiments.

FIG. 7 is a diagram illustrating a configuration of a screen displayed in a display according to one or more embodiments.

FIG. 8 is a flowchart illustrating details of determining a candidate location according to one or more embodiments.

FIG. 9 is a schematic diagram for explaining acquisition of a captured image, acquisition of an indicator, and determination of a candidate position according to one or more embodiments.

FIG. 10 is a schematic diagram illustrating the number of steps, captured images, indices, and candidate flags stored in memory of control device according to one or more embodiments.

FIG. 11A is a schematic diagram illustrating an indicator acquisition procedure in the case of using a root mean square according to one or more embodiments.

FIG. 11B is a schematic diagram illustrating an indicator acquisition procedure in the case of using a standard deviation according to one or more embodiments.

FIG. 12A is a schematic diagram of a graph in a case where three candidate positions are determined without dividing a search range into sections according to a modification example.

FIG. 12B is a schematic diagram of a graph in a case where the search range is divided into three sections and one candidate position is determined for each section according to the modification.

FIG. 13 is a flowchart illustrating details of displaying candidate locations according to one or more embodiments.

FIG. 14 is a flowchart illustrating details of a process of displaying an enlarged image according to one or more embodiments.

FIG. 15 is a schematic diagram for explaining an execute process of imaging a specimen and a process of acquiring a super-resolution image according to one or more embodiments.

FIG. 16A is a flowchart illustrating a process of receiving a selection of a candidate position according to a modification.

FIG. 16B is a flowchart illustrating a process of receiving selection of a candidate position according to the modification.

### DETAILED DESCRIPTION

Hereinafter, an imaging method, a focal position adjustment method, and a microscope system according to one or more embodiments will be described with reference to the drawings. For convenience, orthogonal X, Y, and Z axes may be attached to each other in each drawing. The Z-axis direction is the height direction of the microscope system 1. The X-Y plane is a plane parallel to the horizontal plane. An X-axis positive direction, a Y-axis positive direction, and a Z-axis positive direction are a leftward direction, a forward direction, and an upward direction, respectively.

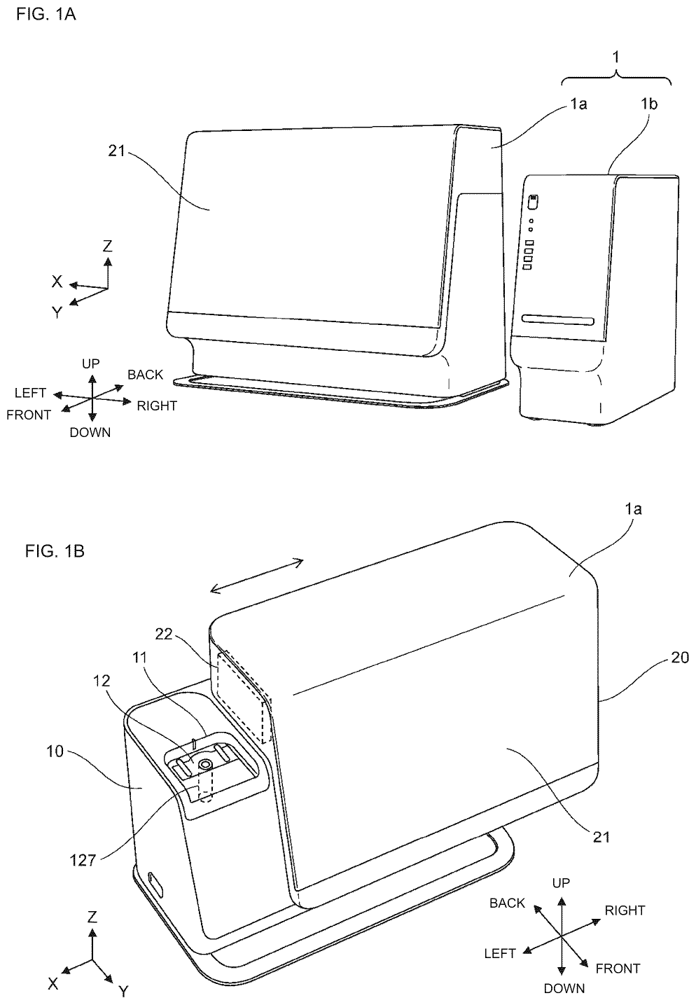

FIG. 1A is a perspective view illustrating a configuration of a microscope system 1.

The microscope system 1 includes a microscope device 1a and a control device 1b. The microscope device 1a and control device 1b are connected to each other by wire for transmitting and receiving signals to and from each other. Note that the microscope device 1a and control device 1b may be connected wirelessly.

The microscope system 1 is a super-resolution microscope device for capturing an image of a specimen and creating and displaying a super-resolution image of the captured specimen. The specimen includes a living body specimen collected from a subject sample (for example, a subject). The biological specimen includes, for example, a protein. The super-resolution microscope is a microscope that observes a subject by a microscopic method that achieves a resolution equal to or lower than the diffraction limit of light, and may have a resolution equal to or lower than 200 nm, which is the limit resolution of a conventional fluorescence microscope. The microscope device 1a may be suitable for observing aggregated proteins in cells including a size of about several tens of nanometers, abnormalities of organelles, and the like.

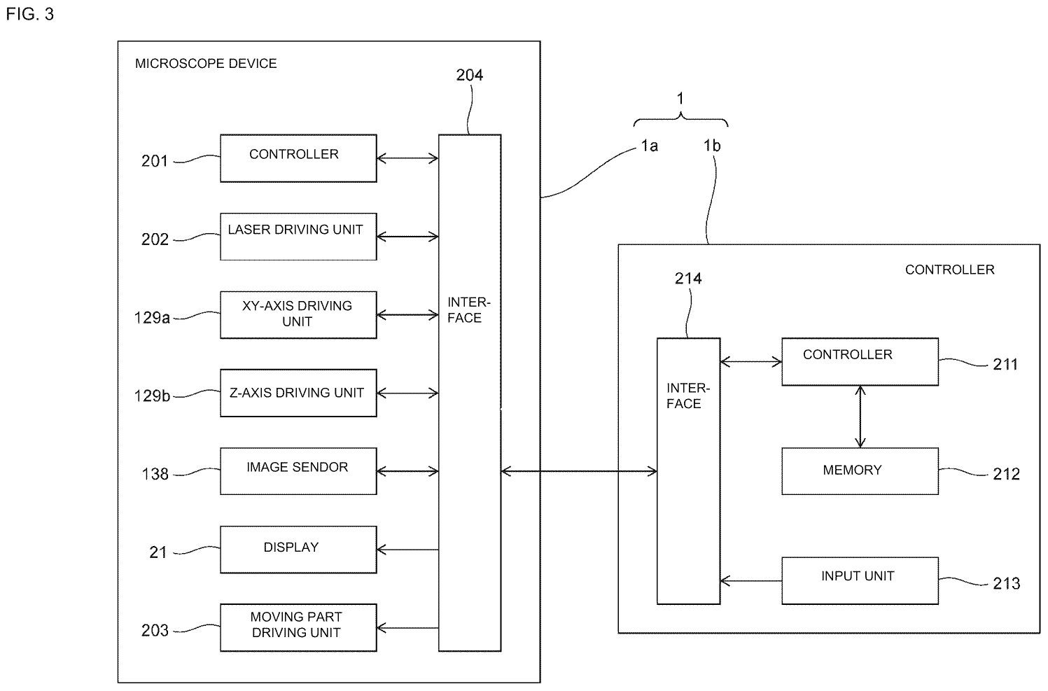

The microscope device 1a includes the display 21 on the front surface. In the display 21, an image related to the imaged specimen is displayed. The control device 1b receives a user instruction via the input unit 213 (see FIG. 3), and controls the microscope device 1a in accordance with a user instruction. The control device 1b processes the image acquired by the microscope device 1a and causes display 21 to display an image related to specimen.

FIG. 1B is a perspective view illustrating the configuration of the microscope device 1a.

The microscope device 1a includes a base unit 10 and a moving part 20.

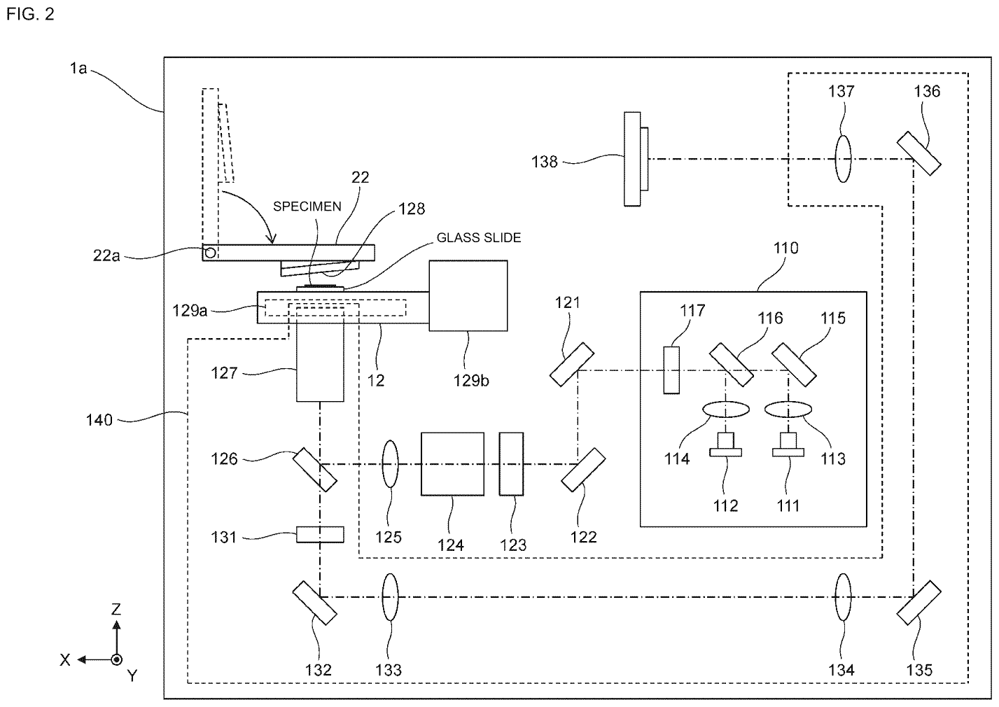

A configuration for imaging specimen (see FIG. 2) is accommodated inside the base unit 10. The recess 11 is formed in an upper portion near the left end of the base unit 10. The specimen setting part 12 is arranged near the bottom surface of recess 11. The specimen setting part 12 is a stage for installing glass slide on which specimen is placed. The objective lens 127 is arranged below the specimen setting part 12.

The moving part 20 is supported by the base unit 10 so as to be movable in the left-right direction between a state of closing the upper side of specimen setting part 12 as shown in FIG. 1A and a state of opening the upper side of specimen setting part 12 as shown in FIG. 1B. As illustrated in FIG. 1B, a user slides the moving part 20 in the right direction to open the upper side of the specimen setting part 12, and places glass slide with specimen in the specimen setting part 12. Subsequently, as illustrated in FIG. 1A, a user slides the moving part 20 in the left direction to close the upper side of the specimen setting part 12. When the upper side of specimen setting part 12 is closed by the moving part 20, the later-described cover 22 provided in the moving part 20 is positioned above specimen setting part 12. Then, a user starts the imaging process by the microscope system 1.

FIG. 2 is a schematical diagram illustrating an internal configuration of the microscope device 1a.

The microscope device 1a includes the first light 110, the mirrors 121 and 122, the filter 123, the beam expander 124, the condenser lens 125, the dichroic mirror 126, the objective lens 127, the second light 128, the specimen setting part 12, the XY-axis driving unit 129a, the Z-axis driving unit 129b, the cover 22, the filter 131, the mirror 132, the imaging lens 133, the relay lens 134, the mirrors 135 and 136, the relay lens 137, and the image sensor 138. The light receiving optical system 140 includes the objective lens 127, the dichroic mirror 126, the filter 131, the mirror 132, the imaging lens 133, the relay lens 134, the mirrors 135 and 136, and the relay lens 137.

The first light 110 includes the light sources 111 and 112, the collimator lenses 113 and 114, the mirror 115, the dichroic mirror 116, and the quarter wavelength plate 117.

The light source 111 emits light of a first wavelength, and the light source 112 emits light of a second wavelength different from the first wavelength. The light sources 111 and 112 are semiconductor laser light sources. The light sources 111 and 112 may be mercury lamps, xenon lamps, LEDs, or the like. The light from the light sources 111,112 is excitation light that causes fluorescence from fluorescent dye coupled to specimen.

The fluorescent dye bound to specimen in advance is a dye that repeats a light emitting state and a light quenching state when irradiated with light of the first wavelength and generates fluorescence when irradiated with light of the first wavelength in the light emitting state. Alternatively, fluorescent dye bound to specimen in advance is a dye that repeats a light emitting state and a light quenching state when irradiated with light of the second wavelength and generates fluorescence when irradiated with light of the second wavelength in the light emitting state. As fluorescent dye, a dye that generates fluorescence having a wavelength that passes through the dichroic mirror 116 and the filter 131 described later is selected. Repetition of a light emitting state and a light quenching state by irradiation with excitation light is referred to as self-destruction, and as fluorescent dye to be self-destructed, for example, SaraFluor 488B and SaraFluor 650B (manufactured by Goryo Chemical, inc.), Alexa Fluor 647 and Alexa Fluor 488 (manufactured by Thermo Fisher Scientific Inc.), and the like may be suitably used.

Either one of the light sources 111 and 112 is used for adjustment of a focal position and acquisition of a super-resolution image, which will be described later, according to fluorescent dye coupled to specimen.

The collimator lenses 113 and 114 respectively collimate the light emitted from the light sources 111 and 112. The mirror 115 reflects the light from the light source 111. The dichroic mirror 116 transmits the light from the light source 111 and reflects the light from the light source 112. A quarter wavelength plate 117 converts linearly polarized light emitted from the light sources 111 and 112 into circularly polarized light. Accordingly, the light emitted from the light sources 111 and 112 may be uniformly absorbed by specimen in any polarization direction. Each unit in the first light 110 is arranged such that optical axes of light from the light sources 111 and 112 emitted from the first light 110 coincide with each other.

The mirrors 121 and 122 reflect the light emitted from the first light 110 to the filter 123. The filter 123 removes light having an unnecessary wavelength out of the light reflected by the mirror 122. The beam expander 124 increases the beam diameter of the light that has passed through the filter 123, and expands the light irradiation region on glass slide installed in specimen setting part 12. As a result, the intensity of the light irradiated onto glass slide is brought close to a uniform state. The condenser lens 125 condenses light from the beam expander 124 so that glass slide is irradiated with substantially parallel light from the objective lens 127.

The dichroic mirror 126 reflects light emitted from the light sources 111 and 112 and collected by condenser lens 125. In addition, the dichroic mirror 126 transmits the fluorescence generated from fluorescent dye coupled to specimen and passed through the objective lens 127. The objective lens 127 guides the light reflected by the dichroic mirror 126 to specimen on glass slide installed in the specimen setting part 12.

The cover 22 is supported by the shaft 22a installed in the moving part 20 (see FIG. 1B) and extending in the Y-axis direction. The cover 22 rotates about the shaft 22a when the moving part 20 moves in the X-axis direction. The cover 22 stands up as shown by broken lines in FIG. 1B and FIG. 2 in conjunction with the movement of the moving part 20 to the right side (X-axis negative direction). When the moving part 20 completely covers the upper side of the specimen setting part 12 by moving the moving part 20 to the left side (X-axis positive direction), the cover 22 rotates by 90 degrees as indicated by a solid line in FIG. 2 in conjunction with this, and the cover 22 becomes parallel to the horizontal plane. The upper side of the specimen setting part 12 is covered with the cover 22.

The second light 128 is provided on a surface of the cover 22 facing specimen setting part 12. The second light 128 is an LED light source that emits white light, and has a planar light-emitting region. The light from the second light 128 is used for capturing a bright field image. The second light 128 is provided to be inclined to the surface of the cover 22. This makes it possible to image specimen with enhanced contrast as compared with the case where the second light 128 is provided parallel to the surface of the cover 22. The structure of the cover 22 that rotates in conjunction with the moving part 20 is disclosed in U.S. Patent Publication No. 2020-0103347, the disclosure of which is incorporated herein by reference.

The specimen setting part 12 is supported in the X-Y plane by the XY-axis driving unit 129a and is supported in the Z-axis direction by the Z-axis driving unit 129b. The XY-axis driving unit 129a includes a stepping motor for moving the specimen setting part 12 in the X-axis direction and a stepping motor for moving the specimen setting part 12 in the Y-axis direction. The Z-axis driving unit 129b includes a stepping motor for moving the specimen setting part 12 and the XY-axis driving unit 129a in the Z-axis direction.

The relative position of the focal point of the light receiving optical system 140 with respect to the specimen setting part 12 changes when the Z-axis driving unit 129b is driven and the specimen setting part 12 moves up and down along the Z-axis. In one or more embodiments, the relative position of the focal point of the light receiving optical system 140 with respect to the specimen setting part 12 is defined by the relative position of the specimen setting part 12 with respect to the objective lens 127. That is, the relative position between the specimen setting part 12 and the focal point of the light receiving optical system 140 is changed by the driving of the Z-axis driving unit 129b, and a plurality of relative positions are generated. When specimen is positioned at the position of the focal point of the light receiving optical system 140, an image in a range of a predetermined viewing angle including the position is formed on the imaging surface of the image sensor 138 by the light receiving optical system 140.

When a fluorescence image is acquired, light is emitted from one of the light sources 111 and 112. When specimen is irradiated with the light from the light source 111 or the light source 112, fluorescence is generated from specimen. The fluorescence generated from specimen passes through the objective lens 127 and is transmitted through the dichroic mirror 126. On the other hand, when a bright field image is acquired, light is emitted from the second light 128. Light from the second light 128 is transmitted through specimen, passes through the objective lens 127, and reaches the dichroic mirror 126. The light in the same wavelength band as that of the fluorescence generated from specimen of the light transmitted through specimen is transmitted through the dichroic mirror 126.

The filter 131 removes light having an unnecessary wavelength out of the light transmitted through the dichroic mirror 126. The mirrors 132,135, and 136 reflect the light transmitted through the filter 131 and guide the light to the image sensor 138. The imaging lens 133 once images the light generated from specimen on the optical path between the imaging lens 133 and the relay lens 134 and guides the light to the relay lens 134. The relay lenses 134 and 137 focus the light generated from specimen on the imaging surface of the image sensor 138. The image sensor 138 is, for example, a CCD image sensor or a CMOS image sensor. The image sensor 138 captures light incident on the imaging surface.

FIG. 3 is a block diagram illustrating a configuration of the microscope system 1.

The microscope device 1a includes the controller 201, the laser driving unit 202, the XY-axis driving unit 129a, the Z-axis driving unit 129b, the image sensor 138, the display 21, the moving part driving unit 203, and the interface 204.

The controller 201 may include a processor such as a CPU or an FPGA, and a memory. The controller 201 controls each unit of the microscope device 1a according to an instruction from control device 1b via the interface 204, and transmits the captured image received from the image sensor 138 to control device 1b.

The laser driving unit 202 drives the light sources 111 and 112 under the control of the controller 201. The XY-axis driving unit 129a includes a stepping motor, and moves the specimen setting part 12 in the X-Y plane by driving the stepping motor under the control of the controller 201. The Z-axis driving unit 129b includes a stepping motor, and moves the XY-axis driving unit 129a and the specimen setting part 12 in the Z-axis direction by driving the stepping motor under the control of the controller 201. The moving part driving unit 203 includes a motor, and moves the moving part 20 in the X-axis positive direction and the X-axis negative direction by driving the motor. The image sensor 138 captures an image of light incident on the imaging surface under the control of the controller 201, and transmits the captured image to the controller 201. The display 21 includes, for example, a liquid crystal display or an organic electroluminescent (EL) display. The display 21 displays various types of screen according to the signal from the control device 1b.

The control device 1b includes the controller 211, the memory 212, the input unit 213, and the interface 214.

The controller 211 includes, for example, a CPU. The memory 212 includes, for example, a hard disk and a solid-state drive (SSD). The input unit 213 includes, for example, a mouse and a keyboard. A user operates the mouse of the input unit 213 to perform an operation such as clicking or double-clicking on screen displayed on the display 21, thereby inputting an instruction to the controller 211. The display 21 and the input unit 213 may be configured by a touch panel display. In this case, a user performs tapping or double tapping on the display surface of the touch panel type display instead of clicking or double clicking.

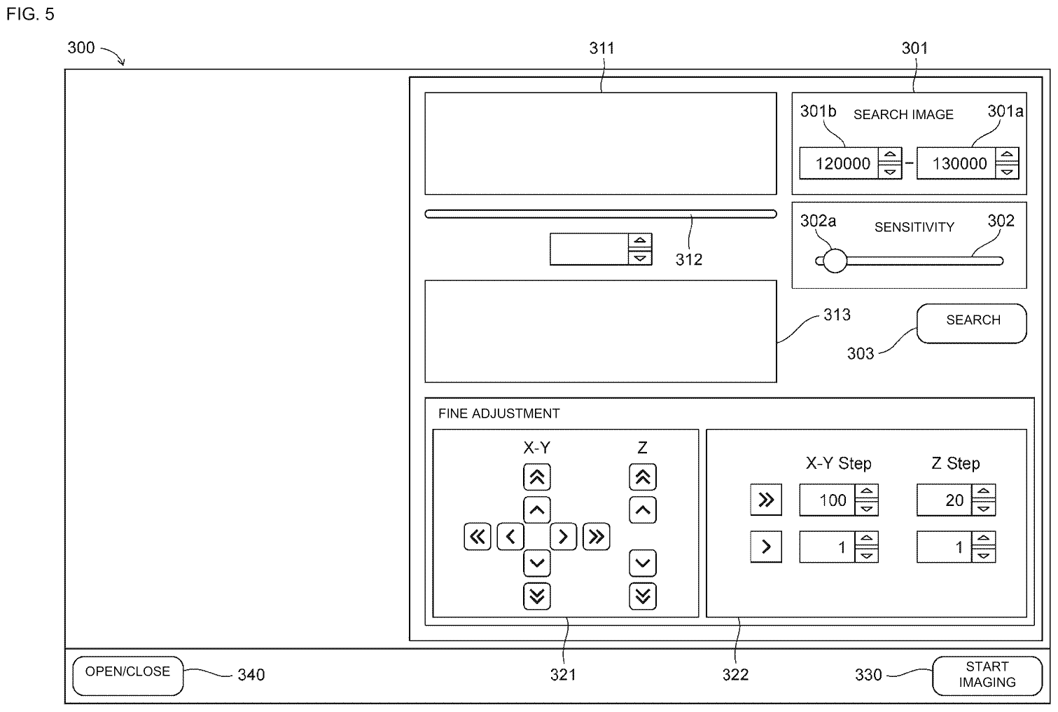

The controller 211 performs processing based on software stored in the memory 212, that is, a computer program and a related file. Specifically, the controller 211 transmits a control signal to the controller 201 of the microscope device 1a via the interface 214, and controls each unit of the microscope device 1a. In addition, the controller 211 receives a captured image from the controller 201 of the microscope device 1a via the interface 214 and stores the captured image in the memory 212. In addition, the controller 211 causes the display 21 of the microscope device 1a to display the screen 300 (see FIGS. 5, 6, and 7) to be described later in order to adjust the focal position based on the captured image received from the microscope device 1a. In addition, the controller 211 causes the microscope device 1a to perform imaging for generating a super-resolution image at the focal position adjusted via the screen 300. The controller 211 generates a super-resolution image based on the captured image and causes the display 21 to display the super-resolution image.

Next, an automatic focus adjustment (autofocus) operation of the microscope system 1 is described.

In an autofocus system of a general microscope, for example, a stripe pattern is projected onto a subject, and the stripe pattern is imaged while changing a relative position between an objective lens and a stage. Then, the software automatically searches for a relative position (focal position) at which the contrast of the image is highest, and the objective lens or the stage is moved to the specified focal position, thereby performing automatic focus adjustment on the subject.

On the other hand, the super-resolution microscope includes a case where the size of the observation target included in specimen is 1 μm or less, for example, a case where the observation target is a protein (10 μm). It is unknown where such an observation target is located in the thickness direction of specimen applied to the slide and what shape the observation target has. Therefore, compared to a case where the shape of the observation target (for example, white blood cell) may be predicted in advance as in, for example, blood smear, in a case where protein is the observation target, it is difficult to increase the accuracy of automatic adjustment of the focal position by software.

Furthermore, in the method of searching for the focal position at which the contrast of the captured image is the highest while changing the focal position, for example, in a case where a bubble included in specimen exhibits higher contrast than the observation target, the focal position may be automatically adjusted in a state where the bubble is in focus. In such a case, a user manually adjusts the focal position without adopting the automatically adjusted focal position. Such an operation takes time. In addition, since fluorescent dye may be deteriorated by being irradiated with light, it may not be preferable to expose fluorescent dye for a long time in order to adjust the focal position before acquiring the super-resolution image.

Therefore, in the microscope system 1 according to one or more embodiments, software does not automatically determine a focal position at one specific position, but specifies a plurality of candidate focal positions based on an indicator obtained from an image and presents the focal positions to a user in a selectable manner. Hereinafter, the processing of the controller 211 according to one or more embodiments is described in detail with reference to a flowchart.

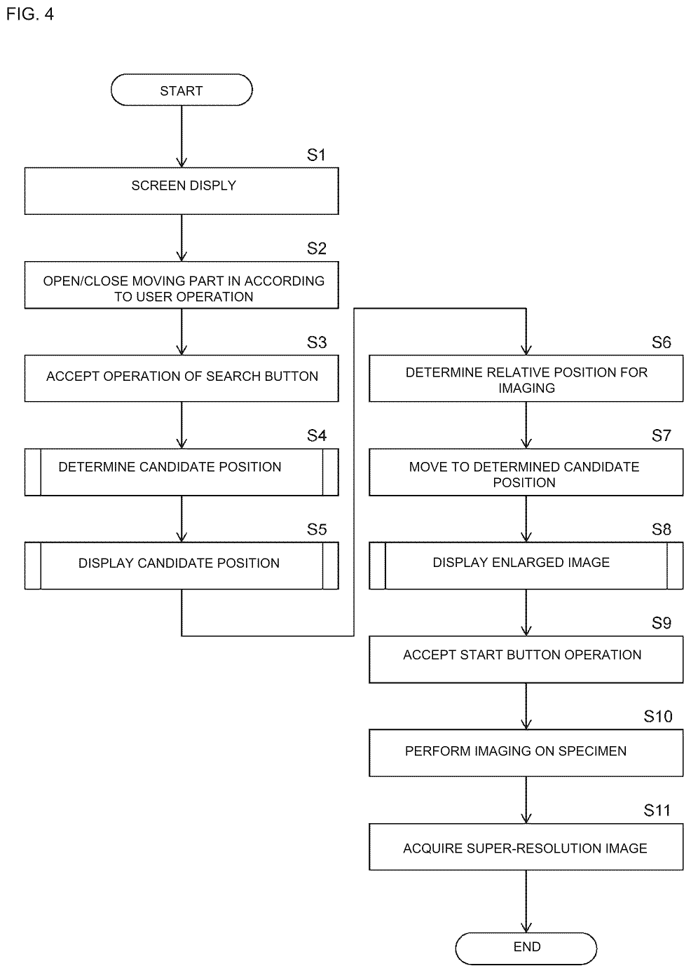

FIG. 4 is a flowchart illustrating processing performed by the controller 211 of control device 1b in the microscope system 1. The controller 211 controls each unit of the microscope device 1a via the controller 201 and the interface 204 of the microscope device 1a.

In step S1, the controller 211 displays the screen 300 (see FIG. 5) on the display 21. The screen 300 will be described later with reference to FIGS. 5-7.

In step S2, the controller 211 opens and closes the moving part 20 in accordance with an operation from a user. When a user operates the open/close button 340 (see FIG. 5) displayed on the screen 300 of the display 21, the controller 211 drives the moving part driving unit 203 to move the moving part 20 in the right direction and expose the specimen setting part 12. A user sets specimen in the specimen setting part 12 exposed. When a user operates the open/close button 340 again, the controller 211 drives the moving part driving unit 203 to move the moving part 20 in the left direction to cover the specimen setting part 12.

In step S3, the controller 211 receives an operation of the search button 303 (see FIG. 5) by a user via the input unit 213. When the search button 303 is operated, in step S4, the controller 211 performs a process of determining a plurality of relative positions (candidate positions) serving as candidates for capturing a super-resolution image.

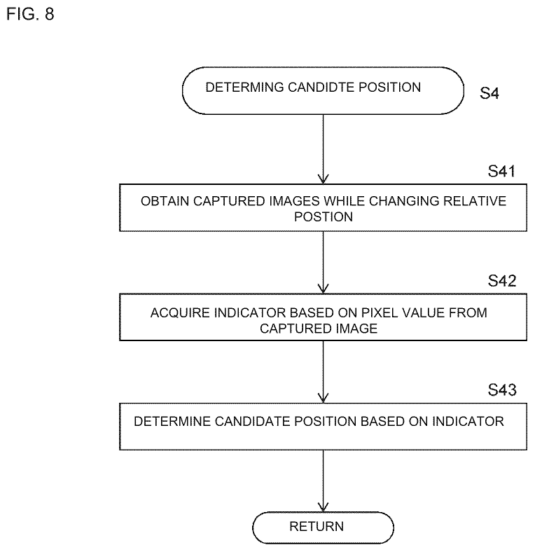

In step S4, the controller 211 drives the Z-axis driving unit 129b to image specimen while changing the relative position between specimen and the objective lens 127. A quantitative indicator for determining whether a subject is in focus is acquired for each of a plurality of captured images obtained by imaging. The controller 211 creates a graph in which the value of the indicator corresponding to each relative position is plotted based on the obtained value of the indicator. The controller 211 determines at least one candidate position for imaging by specifying a plurality of relative positions in descending order of the value of the indicator among the relative positions at which the value of the indicator indicates a peak. Both the relative position and the candidate position are defined by the number of steps from the origin position of the stepping motor of the Z-axis driving unit 129b. Details of step S4 will be described later with reference to FIG. 8.

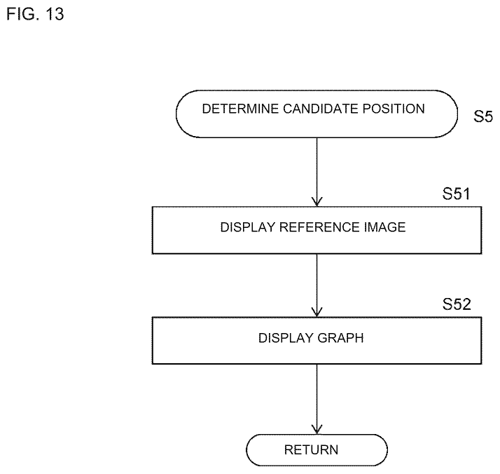

Subsequently, in step S5, the controller 211 displays the candidate positions on the display 21 in a selectable manner. Details of step S5 are described later with reference to FIG. 13.

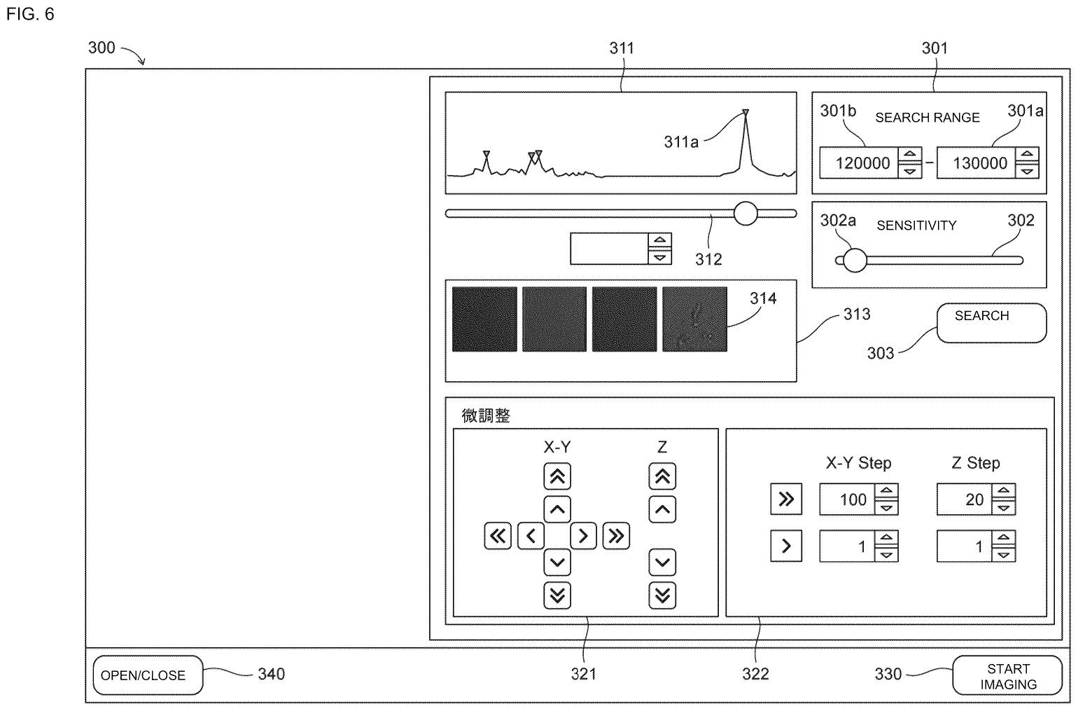

Subsequently, in step S6, the controller 211 determines a relative position for execute imaging based on a user's selection of the candidate position displayed in step S5. Step S6 is described later with reference to FIGS. 5 and 6. In step S7, the controller 211 moves the specimen setting part 12 to the candidate position determined in step S6.

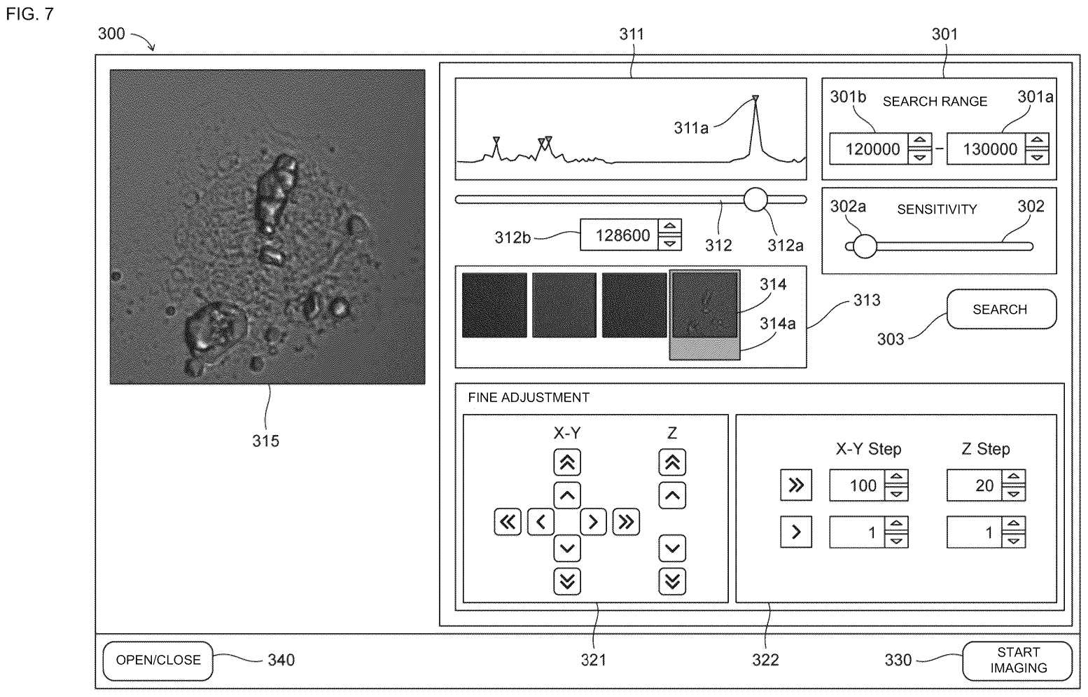

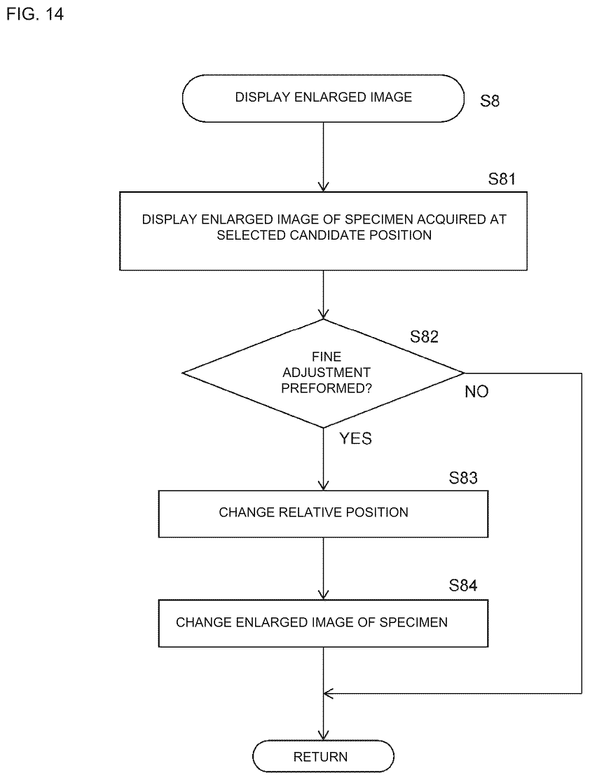

Subsequently, in step S8, the controller 211 the displays the enlarged image. In step S8, the controller 211 displays the enlarged image 315 (see FIG. 7) on the display 21. Details of step S8 are described later with reference to FIG. 14.

Subsequently, in step S9, the controller 211 receives an operation of the start button 330 (see FIG. 7) by a user via the input unit 213.

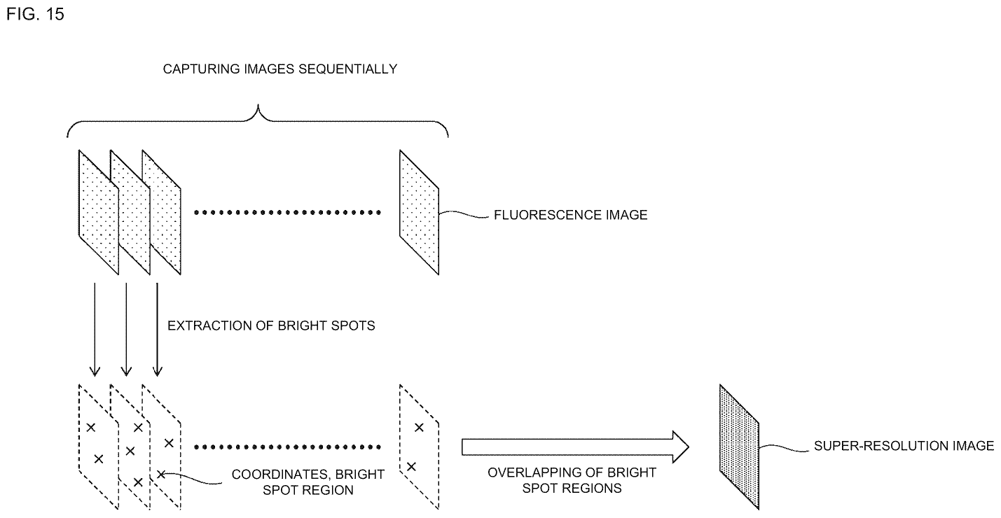

When the start button 330 is operated, in step S10, the controller 211 captures an image of specimen as execute. In step S10, the controller 211 performs execute imaging on specimen at the relative position determined in step S6 or the position of the objective lens 127 finely adjusted in step S83 (see FIG. 14). The controller 211 acquires a plurality of fluorescence images by the image sensor 138 while irradiating specimen with a wavelength set in advance by a user, that is, the first wavelength or the second wavelength. Subsequently, in step S11, the controller 211 acquires a super-resolution image based on the image acquired in step S10. Details of steps S10 and S11 are described later with reference to FIG. 15.

FIGS. 5, 6, and 7 are diagrams illustrating the configuration of the screen 300 displayed on the display 21. FIG. 5 is a diagram illustrating the screen 300 in an initial state, FIG. 6 is a diagram illustrating the screen 300 after the search button 303 is operated, and FIG. 7 is a diagram illustrating the screen 300 after a candidate position is selected.

As illustrated in FIG. 5, the screen 300 includes a search range setting region 301, a sensitivity slider 302, a search button 303, a graph 311, a position slider 312, a reference image area 313, fine adjustment setting areas 321 and 322, and a start button 330.

The search range setting region 301 includes two numerical value boxes 301a and 301b. The search range is a range defined with a first numerical value input to the numerical value box 301a as an upper limit position and a second numerical value input to the numerical value box 301b as a lower limit position. The number of steps of the stepping motor of the Z-axis driving unit 129b corresponding to the upper limit position of the distance between specimen (specimen setting part 12) and the objective lens 127 is input to the numerical value box 301a. The number of steps of the stepping motor of the Z-axis driving unit 129b corresponding to the lower limit position of specimen and the objective lens 127 is input to the numerical value box 301b. The two numerical value boxes 301a and 301b set a range (search range) of the distance between specimen and the objective lens 127 when the captured image is acquired in the process of determining the candidate position.

The sensitivity slider 302 is a slider for setting the interval in the Z-axis direction at which the captured image is acquired in the search range. When the knob 302a of the sensitivity slider 302 is moved to the left, the acquisition interval of the captured image in the Z-axis direction is set to be narrow, and when the knob 302a is moved to the right, the acquisition interval of the captured image in the Z-axis direction is set to be wide. The acquisition interval of the captured image in the search range is defined as, for example, the number of steps of the stepping motor of the Z-axis driving unit 129b per captured image, and maybe set stepwise within a range of, for example, 1 image/10 steps to 1 image/500 steps.

When a user operates the search button 303 after setting glass slide on which specimen is placed in specimen setting part 12, a plurality of captured images are acquired by the image sensor 138 while the relative position between specimen and the objective lens 127 is changed. In one or more embodiments, the relative position between specimen and the objective lens 127 is changed by moving the specimen setting part 12 in one direction along the Z axis with respect to the objective lens 127 whose position is fixed. The captured image thus acquired is stored in the memory 212 of control device 1b. In one or more embodiments, the specimen setting part 12 moves from top to bottom along the Z-axis, but the moving direction may be reversed.

The controller 211 of the control device 1b calculates an indicator to be described later from each of the acquired plurality of captured images. As is described later, the indicator is a numerical value obtained by quantifying the sharpness of an image, which is obtained by performing image analysis on an individual captured image. The larger the numerical value of the indicator is, the clearer the image is, and there is a high possibility that the subject in specimen is in focus. As illustrated in a graph 311 of FIG. 6 to be described later, the controller 211 determines a number Nd (for example, Nd=4) of peaks in descending order of the value of the indicator among a plurality of peaks appearing when the value of the indicator for each position on the Z axis is plotted, and determines a relative position corresponding to each peak as a candidate position for execute imaging. When the search button 303 is operated, the screen 300 changes to the state shown in FIG. 6.

Even after the search button 303 is operated, a user may change the settings of the search range setting region 301 and the sensitivity slider 302 and operate the search button 303 again. Accordingly, the controller 211 performs the acquisition of the captured image, the calculation of the indicator, the determination of the candidate position, and the like again in execute.

As illustrated in FIG. 6, a graph 311 indicates a relationship between the relative position and the value of the indicator acquired for each captured image corresponding to each relative position. The horizontal axis of the graph 311 indicates the relative position, that is, the number of steps applied to the stepping motor of the Z-axis driving unit 129b. The left end of the graph 311 corresponds to the upper limit position of the search range input to the numerical value box 301a, and the right end corresponds to the lower limit position of the search range input to the numerical value box 301b. The vertical axis of the graph 311 indicates the value of the indicator.

A mark 311a in the graph 311 has an arrow shape to indicate positions of points corresponding to the determined four candidate positions. The mark 311a is displayed so as to be selectable by the mouse of the input unit 213. When a user operates the mouse to place the cursor on the mark 311a and clicks the mouse, the mark 311a is selected. An arbitrary point on the graph 311 may be selected by a click operation. From the position of the mark 311a, a user may grasp at which position in the Z-axis direction the value of the indicator corresponding to the candidate position occurs.

The reference image area 313 is a region in which the extracted four captured images are displayed as reference images 314. The reference image 314 in the reference image area 313 is displayed so as to be selectable by the mouse of the input unit 213. When a user operates the mouse to place the cursor on the reference image 314 and clicks the mouse, the reference image 314 is selected.

When a user selects one of the reference image 314 in the reference image area 313 and the mark 311a in the graph 311, a frame 314a appears in the reference image 314 corresponding to the selection result as illustrated in FIG. 7.

In the example illustrated in FIG. 7, when the reference image 314 at the right end among the four reference images 314 or the mark 311a at the right end among the four marks 311a is selected, a frame 314a indicating that the reference image 314 is selected is provided in the reference image 314 at the right end in the reference image area 313. In this case, the knob 312a of the position slider 312 is aligned with the position of the number of steps corresponding to the reference image 314 at the right end, and the value in the numerical value box 312b is the number of steps corresponding to the reference image 314 at the right end. In this manner, the reference image area 313, the graph 311, and the position slider 312 are displayed in conjunction with each other.

In response to selection of the reference image 314 and the mark 311a by a user, the controller 211 determines a candidate position corresponding to the selected reference image 314 or mark 311a as a relative position for execute imaging. the controller 211 applies the number of steps corresponding to the determined relative position to the Z-axis driving unit 129b, thereby moving the specimen setting part 12 to the determined relative position. Then, a captured image is acquired in real time by the image sensor 138. The acquired real-time captured image, that is, the moving image of specimen is displayed as the enlarged image 315 in the screen 300.

After selecting the reference image 314 or the mark 311a via the reference image area 313 and the graph 311 to display the enlarged image 315, a user may finely adjust the relative position using the fine adjustment setting areas 321 and 322.

The fine adjustment setting area 321 includes a plurality of buttons for moving the specimen setting part 12 in the X-axis direction, the Y-axis direction, and the Z-axis direction. Two buttons for movement are provided in one direction. The button labeled “>>” (large movement button) is a button for large movement, and the button labeled “>” is a button for small movement (small movement button). The fine adjustment setting area 322 is provided with numerical value boxes in which a step width as a movement amount corresponding to the large movement button and a step width as a movement amount corresponding to the small movement button may be set. In the example of FIG. 7, for one button operation on the X and Y axes, 100 steps are set as the movement amount of the large movement button, and 1 step is set as the movement amount of the small movement button. For one button operation on the Z axis, 20 steps are set as the movement amount of the large movement button, and 1 step is set as the movement amount of the small movement button.

When the buttons in the fine adjustment setting area 321 are operated, the controller 211 controls the XY-axis driving unit 129a and the Z-axis driving unit 129b according to the number of steps set for each button to move the specimen setting part 12 along the XYZ axes. Even when the specimen setting part 12 is moved, a real-time captured image is acquired by the image sensor 138, and the acquired real-time captured image is displayed as the enlarged image 315.

A user selects a candidate relative position (candidate position) via the reference image 314 and the mark 311a, appropriately adjusts the relative position by the fine adjustment setting areas 321 and 322, and then operates the start button 330 when the position of specimen setting part 12 is determined to be appropriate. As a result, the relative position of specimen setting part 12 at the time when the start button 330 is operated is determined as the relative position for imaging, and imaging for super-resolution image acquisition by the image sensor 138 is performed in this state.

With reference to FIGS. 8, 9, 10, 11A, and 11B, the step of determining the candidate position (step S4 in FIG. 4) will be described.

FIG. 8 is a flowchart showing details of the step of determining a candidate position (step S4 in FIG. 4).

In step S41, the controller 211 of control device 1b images specimen at intervals set by the sensitivity slider 302 while changing the relative position between specimen and the objective lens 127, and acquires a plurality of captured images by the image sensor 138. The captured image acquired in step S41 is an image used to adjust the relative position between specimen and the objective lens 127.

In one or more embodiments, in order to change the relative position, the controller 211 drives the Z-axis driving unit 129b to move the specimen setting part 12 in one direction along the Z-axis. At this time, the movement range of the specimen setting part 12 in the Z-axis direction is the search range set in the search range setting region 301 illustrated in FIG. 5, and the interval at which the captured image is acquired in the Z-axis direction is a distance corresponding to the sensitivity set by the sensitivity slider 302 illustrated in FIG. 5.

At this time, the controller 211 causes any one of the light sources 111 and 112 and the second light 128 to emit light based on the wavelength of the light source selected in advance by a user. Accordingly, when one of the light sources 111 and 112 is driven, the fluorescence generated from fluorescent dye coupled to specimen is imaged by the image sensor 138. When the second light 128 is driven, the light transmitted through the dichroic mirror 116 and the filter 131 in the light transmitted through specimen is imaged by the image sensor 138.

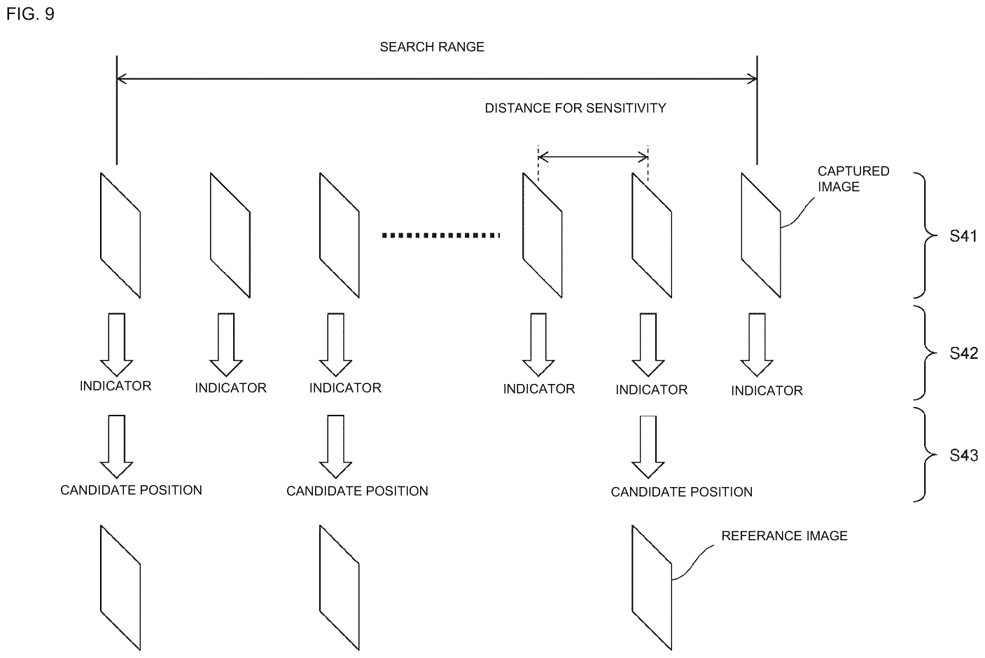

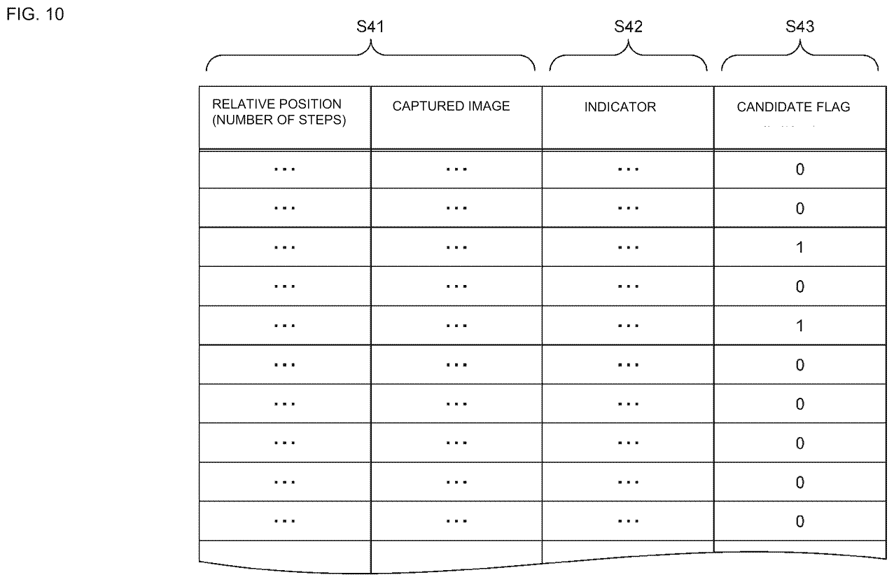

In step S41, when a plurality of captured images are acquired in the search range as shown in FIG. 9, the acquired captured images are stored in memory 212 in association with the relative positions of specimen and the objective lens 127 (the number of steps applied to the stepping motor of the Z-axis driving unit 129b) as shown in FIG. 10. The captured image is stored in memory 212 such that a data file of the captured image is associated with a name and a storage location of the captured image.

Subsequently, in step S42, the controller 211 acquires an indicator based on a pixel value from the captured image acquired in step S41. As a result, as shown in FIG. 9, indices are acquired from all the acquired captured images, and as shown in FIG. 10, the acquired indices are stored in memory 212 in association with the number of steps and the captured images.

Here, a method of acquiring the indicator in step S42 will be described. The method of acquiring the indicator includes a method using a root mean square, a method using a standard deviation, and a method using a contrast.

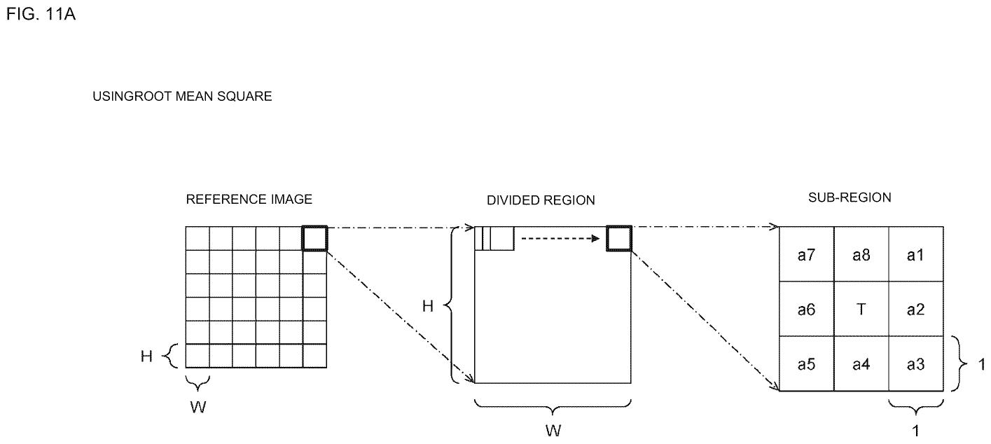

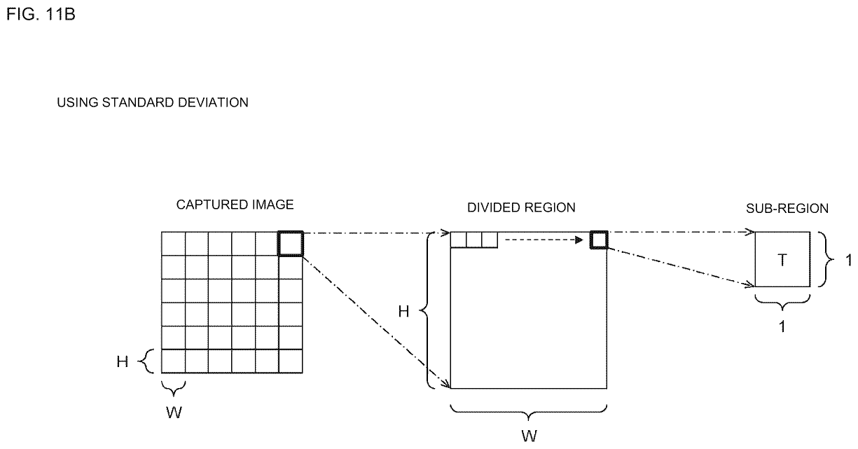

As shown in FIG. 11A, when the root mean square is used, the captured image is equally divided into a predetermined number of divided regions (for example, 36 regions consisting of 6 vertical regions×6 horizontal regions). At this time, the height of one divided region is H and the width thereof is W. The number of divisions of the captured image may be a number other than 36.

Subsequently, in one divided region, a sub-region composed of three dots in the vertical direction and three dots in the horizontal direction around an arbitrary pixel is set. W×H=N sub-regions are provided in one divided region. In the sub-region, when the pixel value of the central pixel is T, the pixel values of the eight pixels located around this pixel are a1 to a8, and the sum of the differences between the pixel value T and the pixel values a1 to a8 is R, the sum R is calculated by the following equation (1).

$$R = {\sum }_{n = 1}^{8} ( T - {a}_{n} ) \qquad ( 1 )$$

Subsequently, while the sub-region is moved by one pixel, the total R is similarly calculated based on the above equation (1) in the N sub-regions in one divided region. When the sum of the i-th sub-regions is _{Ri} and the root mean square in one divided region is RMS, RMS is calculated by the following equation (2).

$$RMS = \sqrt{\frac{1}{N} {\sum }_{i = 1}^{N} {R}_{i}^{2}} \qquad ( 2 )$$

Subsequently, the root mean square RMS is similarly acquired based on the above equation (2) in all the divided regions in the captured image. Then, when the largest value of the root mean square RMS of all the divided regions is RMSmax and the smallest value is RMSmin, the indicator in the case of using the root mean square is calculated by a difference (=RMSmax−RMSmin). The indicator in the case of using the root mean square may be calculated by a ratio (=RMSmax/RMSmin).

As shown in FIG. 11B, when the standard deviation is used, the captured image is equally divided into a predetermined number of divided regions (for example, 36 regions consisting of 6 vertical regions×6 horizontal regions). At this time, the height of one divided region is H and the width thereof is W. The number of divisions of the captured image may be a number other than 36.

Subsequently, in one divided region, a sub-region composed of one vertical dot and one horizontal dot is set. WxH=N sub-regions are provided in one divided region. In N sub-regions in one divided region, when a pixel value of an i-th sub-region is xi, an average value of pixel values of all sub-regions is xa, and a standard deviation in one divided region is σ, σ is calculated by the following equation (3).

$$σ = \sqrt{\frac{1}{N} {\sum }_{i = 1}^{N} {( {x}_{i} - {x}_{a} )}^{2}} \qquad ( 3 )$$

Subsequently, the standard deviation σ is similarly acquired based on the above equation (3) in all the divided regions in the captured image. When the largest value among the standard deviations σ of all the divided regions is σmax and the smallest value is σmin, the indicator in the case of using the standard deviation is calculated by the difference (=σmax−σmin). When the standard deviation is used, the indicator may be calculated by a ratio (=σmax/σmin).

Further, in the case of using the contrast, when the largest pixel value is set as a pixel value max and the smallest pixel value is set as a pixel value min in all pixels of the captured image, the indicator in the case of using the contrast is calculated by a difference (=pixel value max−pixel value min). The indicator in the case of using the contrast may be calculated by a ratio (=pixel value max/pixel value min).

Returning to FIG. 8, in step S43, the controller 211 determines a candidate position based on the indicator acquired from each captured image in step S42. Specifically, the controller 211 specifies a plurality of peaks in a graph indicating the values of the indicators with respect to the positions on the Z axis based on all the values of the indicators acquired for each captured image, determines a number Nd (for example, four) of peak values in descending order based on the value of the indicator (referred to as a peak value) at each peak, and determines a relative position (also referred to as a peak position) corresponding to the determined peak value as a candidate position.

The number Nd may be set to a value other than 4. However, when the number Nd is too small, the number of candidate positions that maybe selected by a user decreases, and a position at which the distance between specimen and the objective lens 127 is appropriate may not be included in the determined candidate positions. On the other hand, when the number Nd is too large, the number of candidate positions to be determined increases, and the burden on a user to select a candidate position increases. Therefore, the number Nd is preferably set in advance in consideration of the balance between these factors. From such a viewpoint, the number Nd is, for example, preferably 2 or more and 20 or less, and more preferably 3 or more and 10 or less.

Alternatively, the search range may be divided into a predetermined number of sections, and the number Nd of peak values may be determined in descending order in each section. For example, the search range may be divided into three sections, and the number Nd of peak values may be determined in descending order in the three sections. In this case, Nd×3 peak values are determined in total, and Nd×3 candidate positions are determined.

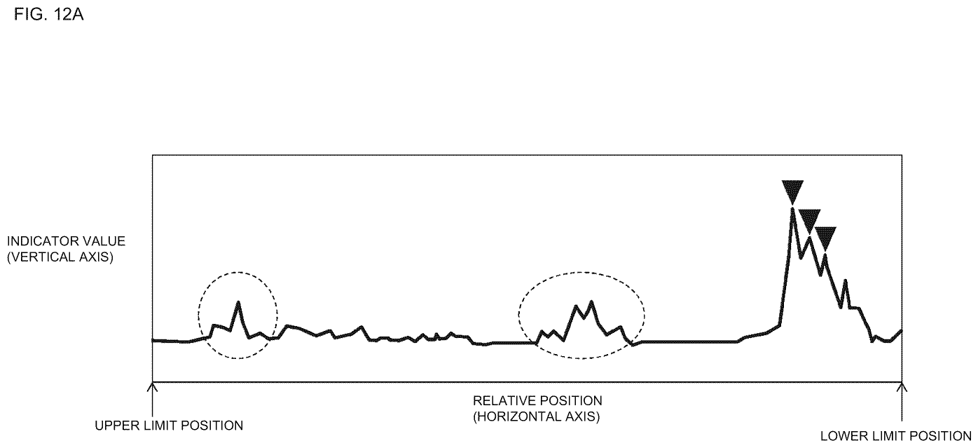

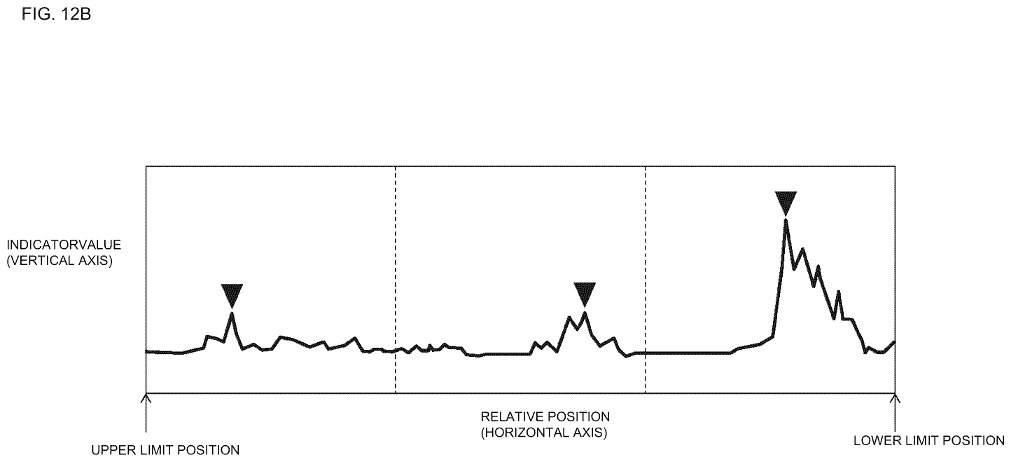

For example, when the number of sections is three and the number Nd of candidate positions determined in each section is two, two candidate positions are determined in descending order of peak value in each section. According to this configuration, the candidate position may be uniformly determined from the entire search range, and oversight of the observation target may be reduced. This will be described in detail with reference to FIGS. 12A and 12B.

FIG. 12A is a schematic diagram of a graph when three candidate positions are determined without dividing the search range into sections. FIG. 12B is a schematic diagram of a graph in a case where the search range is divided into three sections and one candidate position is determined for each section.

As illustrated in FIG. 12A, for example, there may be a case where a plurality of high peaks appear in a concentrated manner in a part of the search range, here, in the vicinity of the lower limit position due to, for example, mixing of air bubbles into specimen, and on the other hand, the relative position when the observation target is focused exists in another part of the search range, for example, the peak surrounded by the broken line. Even in such a case, for example, as illustrated in FIG. 12B, if the search range is divided into a plurality of sections at equal intervals along the Z axis, and a predetermined number of candidate positions are determined for each section, the candidate positions are not localized in a specific part of the search range, and the candidate positions are also determined from other search ranges. Therefore, the possibility that the observation target maybe appropriately detect increased.

Returning to FIG. 8, in step S43, as shown in FIG. 9, the peak values of the number Nd are determined in descending order of the values of all the indicators, and the relative positions corresponding to the determined peak values are determined as the candidate positions. Subsequently, as illustrated in FIG. 10, the controller 211 sets the candidate flag corresponding to the determined indicator to 1, and sets the candidate flag corresponding to the undetermined indicator to 0. As a result, the relative position whose candidate flag is 1 becomes the candidate position. In addition, the captured image and the indicator corresponding to the candidate position are the captured image and the indicator in which the candidate flag is 1.

FIG. 13 is a flowchart illustrating details of the step of displaying candidate positions (step S5 in FIG. 4).

The controller 211 of control device 1b displays the reference image 314 on the screen 300 in step S51, and displays the graph 311 on the screen 300 in step S52. Specifically, as shown in FIG. 10, the candidate position is defined by a candidate flag. The controller 211 displays the captured image whose candidate flag is 1 in the reference image area 313 as the reference image 314. In addition, the controller 211 displays the graph 311 based on the values of all the indicators, and displays a mark 311a indicating the candidate position at the peak to which the candidate flag is set.

The arrangement of the reference image 314 matches the arrangement of the corresponding peaks in the graph 311. For example, the captured image corresponding to the leftmost peak in the graph 311 is displayed on the leftmost side in the reference image area 313, and the captured image corresponding to the rightmost peak in the graph 311 is displayed on the rightmost side in the reference image area 313. This may make it easy to visually grasp the correspondence relationship between the peak in the graph 311 and the reference image 314.

A user refers to the reference images 314 arranged in the reference image area 313, refers to the value of the indicator in the graph 311, and selects a candidate position considered to be most appropriate, that is, an appropriate candidate position where specimen is substantially in focus and there are few bubbles and noise. A user clicks the reference image 314 or the mark 311a corresponding to the selected candidate position via the input unit 213.

In screen example of FIG. 6, four reference images 314 are displayed corresponding to four peaks in the graph 311. Of the four peaks, the rightmost peak shows the highest peak value. A tangible component appears in the reference image 314 displayed on the rightmost side corresponding to the highest peak value, and no tangible component appears in the other reference images 314. If the tangible component shown in the rightmost reference image 314 is the observation target intended by a user, a user may select the reference image 314 or the mark 311a.

In this way, a plurality of candidate positions are determined by one search, and a plurality of reference images 314 corresponding to the plurality of candidate positions are displayed in a selectable manner in a list. For this reason, it may be possible to reduce the trouble of moving the knob 312a of the position slider 312 and searching for a focused image from among a large number of images as in, for example, Patent Document 1 described above. In addition, since not only the captured image having the highest peak value but also the plurality of reference images 314 selected in descending order of peak values are displayed, for example, even in a case where an image of a bubble shows a higher peak value than an image of an observation target, the possibility that a user may select the observation target from the reference images 314 is increased.

If the target observation target does not appear in the plurality of displayed reference images 314, it means that the candidate position where the observation target is in focus is not detect searched this time. In this case, a user may manually move the position slider 312 to search for the observation target, may change the search condition by the search range setting region 301 and the sensitivity slider 302 to perform the search again, or may move the XY coordinate position by the fine adjustment setting area 321 to perform the search.

As described above, even in a case where the observation target is not included in the reference image 314, by displaying the graph 311 as in screen example in FIG. 6, it may be easy for a user to determine an action to be taken next. For example, in screen example of FIG. 6, when the tangible component of the reference image 314 corresponding to the rightmost peak is not the observation target, there is no other peak in which the observation target may appear in the graph 311. Therefore, it is understood that the possibility that the observation target is found is not high even if the position slider 312 is operated. In this case, a user may determine that it is better to change the search condition and search again. On the other hand, in a case where more peaks than in the example of FIG. 6 appear in the graph 311, there may be a possibility that the observation target is reflected in a peak that is not specified as the candidate position, and thus it may be possible to confirm whether the observation target is reflected by operating the position slider 312 or selecting an arbitrary peak of the graph 311.

According to one or more embodiments, it may be possible to reduce the time and effort required for a user to focus on the observation target and shorten the work time required for the focus adjustment. Fluorescent dye may be deteriorated by being irradiated with light, but it may also be possible to avoid exposing fluorescent dye for a long time for focus adjustment.

FIG. 14 is a flowchart illustrating details of the step of displaying the enlarged image (step S8 in FIG. 4).

In step S81, the controller 211 of control device 1b displays, on the screen 300, the enlarged image 315 (see FIG. 7) corresponding to the candidate position determined in step S6 of FIG. 4. As described above, at this point, specimen setting part 12 has been moved to the candidate position determined in step S7 of FIG. 4. Therefore, the controller 211 displays the real-time captured image acquired by the image sensor 138 as the enlarged image 315.

A user refers to the enlarged image 315 to determine whether the selected candidate position is an appropriate position of specimen setting part 12. When a user wants to finely adjust the selected candidate position, a user finely adjusts the position of the specimen setting part 12 via the fine adjustment setting areas 321 and 322 (see FIG. 7).

When the controller 211 receives the fine adjustment of the position of the specimen setting part 12 from a user via the fine adjustment setting areas 321 and 322 (step S82: YES), in step S83, the controller 211 drives the Z-axis driving unit 129b to move the specimen setting part 12 in the Z-axis direction according to the operation of the fine adjustment setting areas 321 and 322. Accordingly, the relative position between specimen and the objective lens 127 is changed. In step S83, the controller 211 drives the XY-axis driving unit 129a to move the specimen setting part 12 in the X-Y plane according to the operation of the fine adjustment setting areas 321 and 322. In step S84, the controller 211 displays the real-time captured image acquired by the image sensor 138 as the enlarged image 315.

When the controller 211 does not receive the fine adjustment from a user (step S82: NO), steps S83 and S84 are skipped. A user may repeat the fine adjustment until the start button 330 is operated.

Thereafter, when the operation of the start button 330 is received in step S9 of FIG. 4, the reception of the selection of the candidate position is completed, and the position of specimen setting part 12 at the time when the start button 330 is operated is determined as the position for imaging. Then, imaging in step S10 is performed at the selected candidate position.

FIG. 15 is a schematic diagram for explaining the processing of steps S10 and S11 in FIG. 4.

In step S10 of FIG. 4, the controller 211 of the control device 1b drives the laser driving unit 202 in a state where the specimen setting part 12 is positioned at the position at the time when the start button 330 is operated, and causes light (excitation light) to be emitted from one of the light sources 111 and 112. A user sets the wavelength of the excitation light corresponding to fluorescent dye coupled to specimen in advance via the input unit 213. The controller 211 causes one of the light sources 111 and 112 to emit excitation light in accordance with the wavelength set by a user. Then, the controller 211 images the fluorescence generated from fluorescent dye bonded to specimen by the image sensor 138.

A fluorescent dye bound to specimen is configured to switch between a light emitting state in which fluorescence is generated and a quenching state in which fluorescence is not generated when the excitation light is continuously irradiated. When fluorescent dye is irradiated with the excitation light, a part of fluorescent dye enters a light emitting state and generates fluorescence. Thereafter, when the excitation light continues to be applied to fluorescent dye, fluorescent dye blinks by itself, and the distribution of fluorescent dye in the light emitting state changes with time. The controller 211 repeatedly images the fluorescence generated while fluorescent dye is irradiated with the excitation light, and acquires several thousands to several tens of thousands of fluorescence images.

In step S11 of FIG. 4, bright spots of fluorescence are extracted by Gaussian fitting for each fluorescence image acquired in step S10. The bright spot is a spot that may be recognized as a bright spot in the fluorescence image. As a result, the coordinates of each bright spot are acquired in the two-dimensional plane. For each fluorescent region in the fluorescence image, when matching with the reference waveform is obtained in a predetermined range by Gaussian fitting, a bright spot region having an area corresponding to this range is assigned to each bright spot. A super-resolution image is created by superimposing the bright spot region of each bright spot obtained in this manner on all the fluorescent images.

According to one or more embodiments, the following effects are achieved.

A plurality of candidate relative positions (candidate positions) are determined based on the captured image obtained while changing the relative position between specimen and the focal point of the light receiving optical system 140 (the number of steps of the Z-axis driving unit 129b) (step S4 in FIG. 4). When the relative position for execute imaging is determined from the plurality of candidate relative positions (step S6 in FIG. 4), specimen imaging is performed at the determined relative position (step S10 in FIG. 4). Thus, a user may adjust the relative position only by selecting a relative position that a user considers appropriate from among the plurality of automatically determined candidate relative positions. Since it is not necessary to find an appropriate relative position from a large number of captured images, it may be possible to adjust the focal position more easily than in the related art.

In the step of displaying an enlarged image (step S8 in FIG. 4), an enlarged image 315 (see FIG. 7) of specimen larger than the reference image 314 (see FIG. 7) is displayed. Accordingly, a user may smoothly determine whether the relative position for execute imaging is appropriate with reference to the enlarged image 315. Further, as illustrated in FIG. 7, since the size of the enlarged image 315 is larger than the size of the reference image 314, a user may more smoothly determine whether the relative position for execute imaging is appropriate by referring to the enlarged image 315.

In the step of displaying candidate positions (step S5 in FIG. 4), a plurality of reference images 314 (see FIG. 6) of specimen corresponding to a plurality of relative positions (candidate positions) serving as candidates are displayed (step S51 in FIG. 13). Thus, when selecting any one of the candidate positions, a user may determine whether the candidate position is appropriate with reference to the reference image 314.

In the step of displaying the candidate position (step S5 in FIG. 4), a plurality of reference images 314 (see FIG. 6) are displayed in a selectable manner, and based on selection of any one of the reference images 314, the relative position corresponding to the selected reference image 314 is determined as the relative position for execute imaging. Accordingly, a user may smoothly select the relative position for execute imaging by performing selection on the reference image 314 while referring to the reference image 314.

The step of determining the relative position for execute imaging (step S6 in FIG. 4) and the step of displaying the enlarged image (step S8) may be repeatedly executed unless the start button 330 (see FIG. 7) is operated. At this time, in the step of displaying the enlarged image, the enlarged image 315 (see FIG. 7) of specimen at the different relative position is displayed according to the determination of the different relative position as the relative position for the imaging of execute. Thus, after determining whether one relative position is appropriate by referring to the corresponding enlarged image 315, a user may smoothly determine whether another relative position is appropriate by referring to the corresponding enlarged image 315.

In the step of displaying the enlarged image (step S8 in FIG. 4), an operation of finely adjusting the relative position for execute imaging is received via the fine adjustment setting areas 321 and 322 in FIG. 7, and the enlarged image 315 (see FIG. 7) is changed according to this operation (step S84 in FIG. 14). Accordingly, a user may smoothly and finely adjust the relative position while referring to the enlarged image 315.

As shown in FIG. 8, the step of determining a plurality of relative positions serving as candidates (candidate positions) (step S4 in FIG. 4) includes acquiring an indicator based on a pixel value from the captured image (step S42) and determining a plurality of relative positions serving as candidates (candidate positions) based on the acquired indicator (step S43). Accordingly, the candidate position may be smoothly acquired from the captured image.

The step of displaying the candidate position (step S5 in FIG. 4) includes a step of displaying the graph 311 (see FIG. 6) indicating the relationship between the plurality of relative positions and the indicator corresponding to each relative position (step S52 in FIG. 13). Accordingly, a user may grasp the relationship between the relative position and the indicator corresponding to the relative position with reference to the graph 311.

In the step of determining the relative position for execute imaging (step S6 in FIG. 4), based on the selection of the relative position via the graph 311 (see FIG. 6), the selected relative position is determined as the relative position for execute imaging. Accordingly, a user may smoothly select the relative position while referring to the graph 311.

In the step of determining a plurality of candidate relative positions (step S4 in FIG. 4), as illustrated in FIGS. 11A and 11B, a pre-indicator is calculated for each divided region obtained by dividing the captured image into a plurality of regions, and an indicator is calculated from the pre-indicators of the plurality of divided regions. At this time, the pre-indicator may be a root mean square, or a standard deviation of values related to pixel values obtained from a plurality of sub-regions in the divided region. According to the indicator calculated using the root mean square or the standard deviation as described above, when the captured image corresponding to the bright field image is acquired, it may be possible to acquire an appropriate candidate position from the captured image.

In the step of determining a plurality of candidate relative positions (step S4 in FIG. 4), the difference or ratio between the maximum value and the minimum value of the pixel values based on the captured image is calculated as an indicator. According to the indicator calculated using the maximum value and the minimum value (contrast) of the pixel values as described above, when the captured image corresponding to the fluorescence image is acquired, an appropriate candidate position may be acquired from the captured image.

In the step of acquiring the super-resolution image (step S11), as described with reference to FIG. 15, the super-resolution image is acquired by processing the image captured in execute step (step S10) for imaging specimen. Since the super-resolution image has a resolution exceeding the diffraction limit of light (about 200 nm), according to the super-resolution image, it may be possible to observe an aggregated protein in a cell having a size of about several tens of nm, an abnormality of an organelle, or the like, and to perform image analysis with high accuracy.

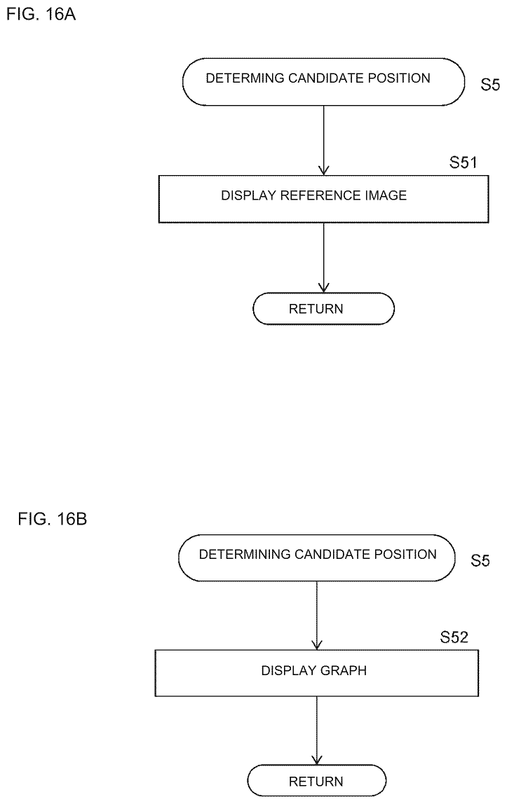

In one or more embodiments, in the step of displaying the candidate positions in FIG. 13, both the display of the reference image 314 (step S51) and the display of the graph 311 (step S52) are performed. However, the scope is not limited thereto, and only the reference image 314 may be displayed as illustrated in FIG. 16A, or only the graph 311 may be displayed as illustrated in FIG. 16B.

In the step (step S6) of determining the relative position for execute imaging in FIG. 4, the candidate position is selected via the reference image 314 and the mark 311a. However, the scope is not limited thereto, and the candidate position may be selected only through the reference image 314, or the candidate position may be selected only through the mark 311a. The candidate position may be selected by operating the knob 312a or the numerical value box 312b of the position slider 312.

In one or more embodiments, the relative position between specimen and the objective lens 127 is changed by changing the position of the specimen setting part 12 in the Z-axis direction between the specimen setting part 12 and the objective lens 127. However, the relative position between specimen and the objective lens 127 may be changed by changing the position of the objective lens 127 in the Z-axis direction. In this case, the number of steps of a stepping motor of a Z-axis driving unit separately provided to drive the objective lens 127 in the Z-axis direction corresponds to the relative position between specimen and the objective lens 127. The relative positions may be changed by changing the positions of both the specimen setting part 12 and the objective lens 127 in the Z-axis direction.

Furthermore, the relative position between specimen and the focal point of the light receiving optical system 140 may be adjusted by moving an optical element other than the objective lens 127 in the light receiving optical system 140. For example, an inner focus lens may be provided in addition to the objective lens 127, and the focus of the light receiving optical system 140 may be changed by moving the inner focus lens. In this case, the candidate position determined in step S4 of FIG. 4 is acquired as the position of the inner focus lens.

In one or more embodiments, the candidate position determined in step S4 of FIG. 4 is the number of steps corresponding to the drive position of the stepping motor of the Z-axis driving unit 129b. However, the candidate position is not limited thereto, and may be a value that uniquely determines the relative position between specimen and the objective lens 127. For example, in the case according to one or more embodiments, the distance may be a distance indicating how much specimen setting part 12 has moved in the Z-axis direction from the origin.

In one or more embodiments, in step S43 of FIG. 8, the value of the indicator of the number Nd in descending order of all the peak values is determined as the value corresponding to the candidate position. However, the scope is not limited thereto, and a value of an indicator that is equal to or greater than the threshold Th among the values of all the indicators may be determined as the value corresponding to the candidate position. In this case, even if step S43 is executed, there may be a case where no candidate position is listed. For example, in a case where pretreatment for specimen is not appropriate, or in a case where the placement of specimen or the installation of glass slide is not appropriate, there is no indicator that is equal to or greater than the threshold Th, and the candidate position may not be listed.

In one or more embodiments, as illustrated in FIG. 8, in the step of determining the candidate position, the controller 211 acquires all the captured images, then acquires the indices from all the captured images, and determines the candidate position based on the acquired indices. However, the scope is not limited thereto, and the controller 211 may acquire the captured image while changing the relative position between specimen and the objective lens 127 and acquire the indicator from the acquired captured image. In this case, if the value of the indicator sequentially acquired in accordance with the captured image is equal to or greater than the threshold Th, the controller 211 determines the value of the indicator as the value corresponding to the candidate position.

In one or more embodiments, the enlarged image 315 displayed by selecting the candidate position is a real-time image acquired by the image sensor 138. However, the scope is not limited thereto, and the enlarged image 315 may be a still image. For example, the enlarged image 315 may be an image obtained by enlarging the captured image corresponding to the selected candidate position, in other words, the captured image displayed as the reference image 314.

When the reference image 314 is displayed as the enlarged image 315, the controller 211 moves the specimen setting part 12 in step S83 of FIG. 14, and then displays the captured image of the still image acquired by the image sensor 138 as the enlarged image 315.

In one or more embodiments, among the captured images captured in step S41 of FIG. 8, the captured image whose candidate flag is 1 is displayed as the reference image 314. However, the scope is not limited thereto. After the candidate position is determined in step S43, the specimen setting part 12 may be moved based on the candidate position, a captured image corresponding to the candidate position may be captured again, and the acquired captured image may be displayed as the reference image 314.

In one or more embodiments, the reference image 314 displayed in FIGS. 6 and 7 may be selected according to an operation on the reference image 314. However, the scope is not limited thereto, and the reference image 314 may be selected by a button, a check box, or the like added to the reference image 314.

In one or more embodiments, in the process of determining the candidate position (step S4 in FIG. 4), the indicator based on the pixel value is acquired from the plurality of captured images, and the candidate position at which the relative distance between specimen and the objective lens 127 is considered to be appropriate is determined based on the acquired indicator. However, the method of determining at least one candidate position by analyzing a plurality of captured images is not limited thereto. For example, a plurality of captured images may be analyzed by a deep learning algorithm to select the captured images, and a candidate position corresponding to the selected captured image may be determined. Also in this case, a user may set the relative position between specimen and the objective lens 127 to an appropriate position by selecting any one of the candidate positions determined by the deep learning algorithm.

One or more embodiments may be variously modified as appropriate within the scope of the technical idea described in the claims.

In the adjustment method of Patent Document 1, a user needs to select a phase image in the imaging state from phase images while moving the knob of the slider, which may be complicated.

According to the imaging method for imaging specimen of the microscope system, the focal position adjustment method for adjusting the focal position of the microscope system, and the microscope system according to one or more embodiments, the focal position for specimen may be set more easily than in the related art.

As a supplementary note, an imaging method, a focal position adjusting method, and a microscope system are summarized.

An imaging method of imaging a specimen using a microscope system, the method comprising:
determining candidate relative positions based on captured images of the specimen, the images being obtained by capturing images of while changing a relative position between the specimen and a focal point of a light receiving optical system of the microscope system;
determining a relative position for capturing among the candidate relative positions; and
capturing an image of the specimen at the determined relative position.

In the imaging method, the method further comprises
displaying an image of the specimen corresponding to the relative position for capturing.

In the imaging method, the method further comprises
displaying reference images of the specimen corresponding to the candidate relative positions.

In the imaging method, the method further comprises
displaying reference images for selecting, wherein
based on selecting a reference image among the displayed reference images, a relative position corresponding to a selected reference image is determined as the relative position for capturing.

In the imaging method, the method further comprises
displaying an enlarged image of the specimen that is captured at the relative position and that is larger than the reference image.

In the imaging method, the displaying the enlarged image comprises
displaying the enlarged image of the specimen at a different relative position, in response to the different relative position being determined as the relative position for capturing.

In the imaging method, the displaying the enlarged image comprises:
receiving an operation of finely adjusting a relative position for capturing; and changing the enlarged image according to the operation.

In the imaging method, the determining the candidate relative positions comprises:
obtaining an indicator based on a pixel value from the captured image; and
determining the candidate relative positions based on the indicator.

In the imaging method, the method further comprises
displaying a graph showing a relationship between the relative positions and the indicators corresponding to the relative positions.

In the imaging method, the relative position for capturing is determined by selecting a relative position in the graph.

In the imaging method, the determining the candidate relative positions comprises:
calculating pre-indicators for divided regions obtained by dividing the captured image; and
obtaining the indicator based on the pre-indicators of the divided regions.

In the imaging method, the divided regions include regions obtained by equally dividing the captured image.

In the imaging method, the pre-indicators comprise a root mean square or a standard deviation of values for pixel values obtained from sub-regions within the divided region.

In the imaging method, the indicator includes a difference or a ratio between a maximum value and a minimum value of pre-indicators of the regions.

In the imaging method, the determining the candidate relative positions comprises calculating a difference or a ratio between a maximum value and a minimum value of the pixel values based on the captured image as the indicator.

In the imaging method, the method further comprises
obtaining a super-resolution image to process the captured image of the specimen.

A method of focal position adjusting that adjusts a relative position between a specimen and a focal point of a light receiving optical system using a microscope system, the method comprising:
determining candidate relative positions based on captured images of the specimen, the images being obtained by capturing specimen images while changing the relative position between specimen and the focal point of the light receiving optical system of the microscope system; and
determining a relative position for capturing among the candidate relative positions.

A microscope system that captures a specimen image comprising:
a specimen setting part that is placed specimen;
an image sensor that captures an image of the specimen through a light receiving optical system;
a driving unit that changes a relative position of a focal point of the light receiving optical system with respect to the specimen setting part; and
a controller that performs operations comprising:
determining candidate relative positions based on specimen images captured by the image sensor while changing the relative position;
determining a relative position for capturing from the relative positions; and
capturing a specimen image at the relative by the image sensor.

1. An imaging method of imaging a specimen using a microscope system, the method comprising:
determining candidate relative positions based on captured images of the specimen, the images being obtained by capturing images while changing a relative position between the specimen and a focal point of a light receiving optical system of the microscope system;
determining a relative position for capturing among the candidate relative positions; and
capturing an image of the specimen at the determined relative position.

2. The imaging method according to claim 1, further comprising
displaying an image of the specimen corresponding to the relative position for capturing.

3. The imaging method according to claim 1, further comprising
displaying reference images of the specimen corresponding to the candidate relative positions.

4. The imaging method according to claim 3, further comprising
displaying reference images for selecting, wherein
based on selecting a reference image among the displayed reference images, a relative position corresponding to a selected reference image is determined as the relative position for capturing.

5. The imaging method according to claim 3, further comprising
displaying an enlarged image of the specimen that is captured at the relative position and that is larger than the reference image.

6. The imaging method according to claim 5, wherein
the displaying the enlarged image comprises displaying the enlarged image of the specimen at a different relative position, in response to the different relative position being determined as the relative position for capturing.

7. The imaging method according to claim 5, wherein
the displaying the enlarged image comprises:
receiving an operation of finely adjusting a relative position for capturing; and
changing the enlarged image according to the operation.

8. The imaging method according to claim 1, wherein
the determining the candidate relative positions comprises:
obtaining an indicator based on a pixel value from the captured image; and
determining the candidate relative positions based on the indicator.

9. The imaging method according to claim 8, further comprising
displaying a graph showing a relationship between the relative positions and the indicators corresponding to the relative positions.

10. The imaging method according to claim 9, wherein
the relative position for capturing is determined by selecting a relative position in the graph.

11. The imaging method according to claim 8, wherein
the determining the candidate relative positions comprises:
calculating pre-indicators for divided regions obtained by dividing the captured image; and
obtaining the indicator based on the pre-indicators of the divided regions.

12. The imaging method according to claim 11, wherein
the divided regions include regions obtained by equally dividing the captured image.

13. The imaging method according to claim 11, wherein
the pre-indicators comprise a root mean square or a standard deviation of values for pixel values obtained from sub-regions within the divided region.

14. The imaging method according to claim 11, wherein
the indicator includes a difference or a ratio between a maximum value and a minimum value of pre-indicators of the regions.

15. The imaging method according to claim 8, wherein
the determining the candidate relative positions comprises calculating a difference or a ratio between a maximum value and a minimum value of the pixel values based on the captured image as the indicator.

16. The imaging method according to claim 1, further comprising
obtaining a super-resolution image to process the captured image of the specimen.

17. A method of focal position adjusting that adjusts a relative position between a specimen and a focal point of a light receiving optical system using a microscope system, the method comprising:
determining candidate relative positions based on captured images of the specimen, the image being obtained by capturing specimen image while changing the relative position between specimen and the focal point of the light receiving optical system of the microscope system; and
determining a relative position for capturing among the candidate relative positions.

18. A microscope system that captures a specimen image comprising:
a specimen setting part that is placed specimen;
an image sensor that captures an image of the specimen through a light receiving optical system;
a driving unit that changes a relative position of a focal point of the light receiving optical system with respect to the specimen setting part; and
a controller that performs operations comprising:
determining candidate relative positions based on specimen images captured by the image sensor while changing the relative position;
determining a relative position for capturing from the relative positions; and
capturing a specimen image at the relative by the image sensor.
